(19) United States
(12) Patent Application Publication
(10) Pub. No.: US 2009/0156468 A1
(43) Pub. Date: Jun. 18, 2009

(54) Use of human chorionic gonadotropin oral or injectable for metabolic syndrome treatment

(75) Inventor: Daniel Oscar Belluscio, Capital Federal (AR)

Correspondence Address: BELLUSCIO, DANIEL OSCAR, ESTADOS UNIDOS 972 10 E, CAPITAL FEDERAL / BUENOS AIRES, 1101, AR

(73) Assignee: Dr. Daniel Oscar Belluscio, Capital Federal (AR)

(21) Appl. No.: 12/314,719

(22) Filed: Dec. 16, 2008

(30) Foreign Application Priority Data
Dec. 7, 2007 (AR) P20070105510

(51) Int. Cl. A61K 38/16

(52) U.S. Cl. 514/8

(57) ABSTRACT

To be used as a therapy for patients with one or more of the following clinical symptoms or laboratory findings: high blood pressure, diabetes type 2, reactive hyperglycemia, plasmatic hypertriglyceridemia, hypercholesterolemia and gout as part of the metabolic syndrome.

### DETAILED DESCRIPTION OF INVENTION

This invention relates to “THE USE OF HUMAN CHORIONIC GONADOTROPIN ORAL OR INJECTABLE FOR THE TREATMENT OF METABOLIC SYNDROME”. The use of Human Chorionic Gonadotropin oral or injectable for the treatment of metabolic syndrome.

### SCOPE

To be used as a therapy for patients with one or more of the following clinical symptoms or laboratory findings: high blood pressure, diabetes type 2, reactive hyperglycemia, plasmatic hypertriglyceridemia, hypercholesterolemia, and gout as part of the metabolic syndrome.

### CURRENT TECHNIQUE

Metabolic Syndrome (MS), also known as plurimetabolic syndrome, insulin-resistant syndrome or syndrome X, is a clinical entity which, with broad phenotypic variations, is suffered by individuals with endogenous predisposition. to it as genetically determined and conditioned by environmental factors.

Typically, it shows insulin resistance and compensatory hyperinsulinemia associated with hydrocarbonated metabolic disorders, high blood pressure, lipid alterations (hypertriglyceridemia, decreased HDLC, the presence of LDL type-B, increased free fatty acids and postprandial lipemia) and obesity, resulting in an increase in morbidity and mortality due to atherosclerosis.

There are other factors associated with MS:
hyperuricemia or gout;
Thrombophilia and fibrinolysis defects;
Hyperleptinemia or leptin resistance; and also: homocysteine (controversial role in IR), leukocytosis, increased GSR, increased PAI-1, hyperandrogenism, fatty liver, gallstones, osteoporosis, Acanthosis Nigricans, and polycystic ovary syndrome.

Many cases evidence that diabetes mellitus (DM) is potentially more frequent in MS patients (A).

MS is the aggregate of the most dangerous heart-risk factors, i.e., diabetes and pre-diabetes, abdominal obesity, changes in cholesterol rate and high blood pressure.

Although 80% of the nearly 200 million diabetic adults worldwide will die due to heart disease, MS subjects are also in a greater-risk sage and are twice as potential sufferers of heart arrest or heart attack as the rest of the persons who do not suffer this syndrome.

Due to this fact, MS and diabetes are ranked above AIDS/HIV with relation to mortality and morbidity, even though it has not the same level of recognition as the latter.

MS persons have five times more probabilities to develop diabetes 2 (unless they have it already).

This the real aggregate of the additional risk factors expected from each of the components (ex., the appearance of high levels of triglycerides on cholesterol test).

From a practical and essentially medical perspective, the parameters proposed above are the most extended for MS identification, as shown in the following table in a simple way:

| | Men | | Women | |
|---|---|---|---|---|
| Abdominal obesity (waist | >102 | cm | >88 | cm |
| circumference) | | | | |
| Triglycerides | ≧150 | mg/dl | ≧150 | mg/dl |
| Hypercholesterolemia | <40 | mg/dl | <50 | mg/dl |
| Blood pressure | ≧130/≧85 | mmHg | ≧130/≧85 | mmHg |
| Fast glycemia | ≧110 | mg/dl | ≧110 | mg/dl |

Diagnosis is established where three or more of the risk determinants above are present.

Forty percent of population at large may be prone to IR.

MS affects 42% of women and 64% of men who are glucose-intolerant, and 78% of women and 84% of men with DM2.

MS triples the risk for heart disease (up to 80% MS patients die because of complications of heart disease).

MS is also associated with global increase in mortality for any cause.

### RELEVANCE IN ARGENTINA

According to studies performed, MS relevance is high, with percentages of about 30% of population at large.

With relation to the different definition criteria mentioned above, MS prevalence was 34.9%, 27.2% and 25.6%, respectively, and more frequent in men than women—39.2% vs. 29.0%—respectively.

MS prevalence increased with age, but there were no significant differences between men and women from 60 to 65 years old (39.2% increase in men and 39.1% in women).

Once adjustments were made according to age, sex, physical activities, history of diabetes and menopausia, low-education level subjects had 54% higher risk for MS and 44% higher risk for “hypertriglyceridemic waist” (defined as simultaneous presence of central obesity [in this paper determined under IDF criterion] and triglycerides >150 mg/dl) compared to high-education level subjects.

MS high prevalence levels show the importance of detection and treatment. Low education level was an independent predictor, and this social class should have a priority concerning heart disease and diabetes prevention.

### PREDICTION OF MS EVOLUTION

Average follow up for 8.9 years showed that mortality due to heart disease increased separately in 45% of men and 73% of women with MS. MS total mortality relative risk was 27% in men and 25% in women. Therefore, there is an urgent need to use medical treatment to improve life quality in these patients.

### PROBLEM TO BE SOLVED

Regarding treatment, currently there are no alternatives providing for the management of MS patients with a single medicine.

### THIS INVENTION CAN SOLVE THE PROBLEM

The new application of Human Chorionic Gonadotropin as established on this invention is highly relevant since it enables the improvement of several clinical parameters through a single therapeutic alternative.

### ADVANTAGES

The use of Human Chorionic Gonadotropin oral or for injectable together with a very low-calorie diet for a short period for the treatment of MS under a precise control and follow-up protocol allows simultaneous treatment of the following symptoms:
1) Hyperglycemia
2) Hypertension
3) Hypertriglyceridemia
4) Abdominal obesity
5) Sleep apnea that is frequently found as part of the MS

### DESCRIPTION OF INVENTION

“THE USE OF HUMAN CHORIONIC GONADOTROPIN ORAL OR INJECTABLE FOR THE TREATMENT OF METABOLIC SYNDROME” under clinical control, wherein patients are administered Chorionic Gonadotropin (hCG) orally or by injection.

Daily doses of Gonadotropin are adjusted depending on the type of overweight to 300-600 International Units per day (for oral administration, patients maintain Gonadotropin solution for 1-2 minutes in their mouth for an easier absorption by the rich venous plexus of the mouth, and then swallow it) or 100-300 IU (by IM injection) during the whole treatment period.

Moreover, patients have to follow a very low-calorie diet (about 500 Kcal/day) that is also low-fat, hypohydrocarbonate and normoproteic and provides 200 gr of animal protein plus a combination of vegetables and carbohydrates up to completion of the necessary calories.

The treatment takes at least one month and can be extended up to two months. Then there follows a one-month period to maintain weight, after which the treatment can be repeated.

No hCG treatment is followed at intervals, but a usual hypohydrocarbonate diet is prescribed.

A combined therapy of hCG+very low-calorie diet, due to its action on fatty tissue inhibiting its synthesis, and due to its action on the hypothalamus, results in:

1. Constant hyperglycemia during the treatment period.

2. Fast improvement of hypertriglyceridemia.

3. Reduction of cholesterol high levels.

4. Stabilization of blood pressure to normal or acceptable levels.

5. Marked reduction of total fat mass.

6. A feeling of well being during the treatment period.

7. Reduction of abdominal diameter.

The advantages of this invention, which should not be limited to the brief description above, will become more apparent and the invention itself better understood by reference to the following cases of patients treated using the method described above, a conclusion of the treatment, and the figures of comparative tables showing body weight evolution, abdomen measurement and glycemia tests.

(FIGS. 1, 2, 3, 4, 5, 6, 7, 8 and 9).

⊚Patient AB (FIGS. 1, 2 and 3)

Male aged 38 years, with the following MS parameters:
Weight: 115.700 Kg
Height: 1.74 cm
Abdominal obesity (waist circumference): 122 cm
Triglycerides: 250 mg/dl
HDLC: 38 mg/dl
Blood pressure: 140-90 mmHg (administered with antihypertensive medication)
Fast glycemia: 184 mg/dl (administered with oral antidiabetic medication)

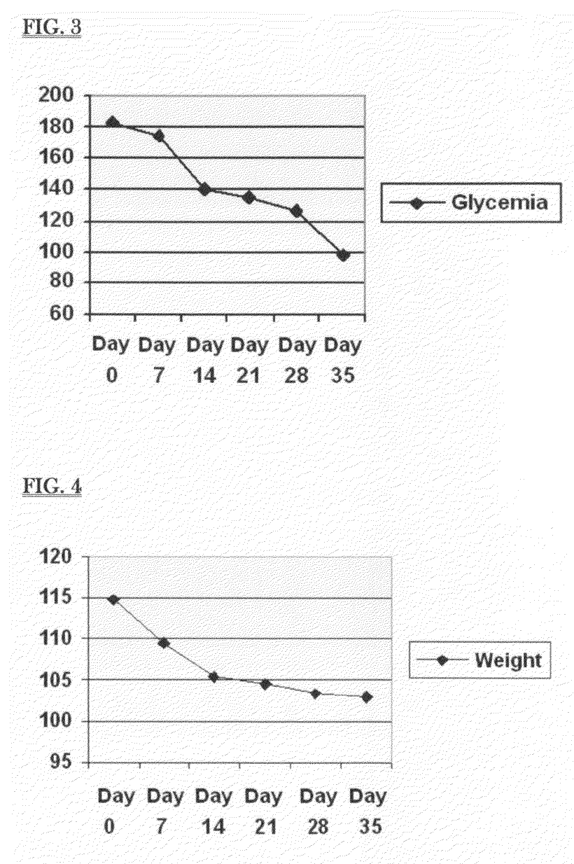

⊚Patient AF (FIGS. 4, 5 and 6)

Male aged 59 years, with the following MS parameters:
Weight: 114.800 Kg
Height: 1.86 cm
Abdominal obesity (waist circumference): 132 cm
Triglycerides: 313 mg/dl
HDLC: 32 mg/dl
Blood pressure: 160-100 mmHg (administered with antihypertensive medication)
Fast glycemia: 255 mg/dl (administered with oral antidiabetic medication)

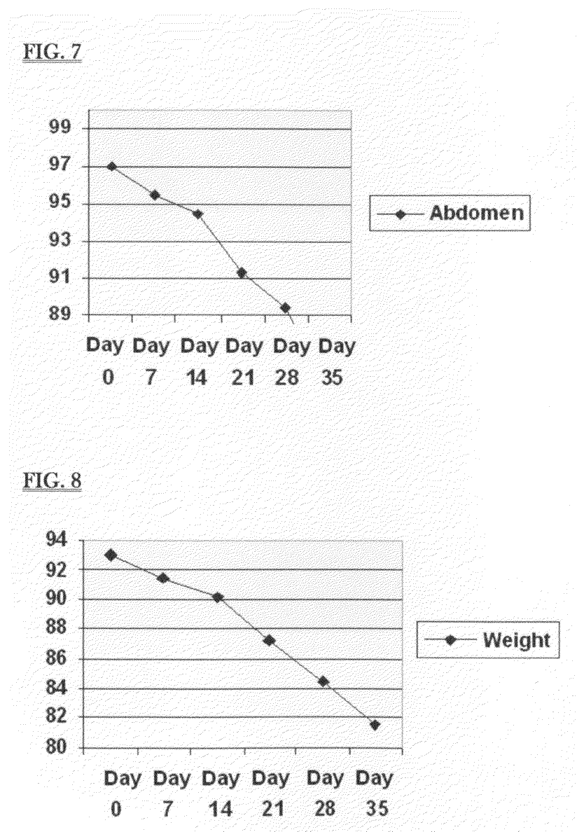

⊚Patient LVM (FIGS. 7, 8 and 9)

Female aged 60 years, with the following MS parameters:
Weight: 93 Kg
Height: 1.61 cm
Abdominal obesity (waist circumference): 101 cm
Triglycerides: 290 mg/dl
HDLC: 43 mg/dl
Blood pressure: 170-100 mmHg (administered with antihypertensive medication)
Fast glycemia: 194 mg/dl (administered with oral antidiabetic medication)

The evolution of these patients during the treatment period with THE USE OF HUMAN CHORIONIC GONADOTROPIN ORAL OR INJECTABLE FOR THE TREATMENT OF METABOLIC SYNDROME is detailed below together with the Figures attached.

First patient:

Patient AB: male

Male aged 38 years, with the following MS parameters:
Weight: 115.700 kg
Height: 1.74 cm
Abdominal obesity (waist circumference): 122 cm
Triglycerides: 250 mg/dl
HDLC: 38 mg/dl
Blood pressure: 140-9 0 mmHg (administered with antihypertensive medication)
Fast glycemia: 184 mg/dl (administered with oral antidiabetic medication)

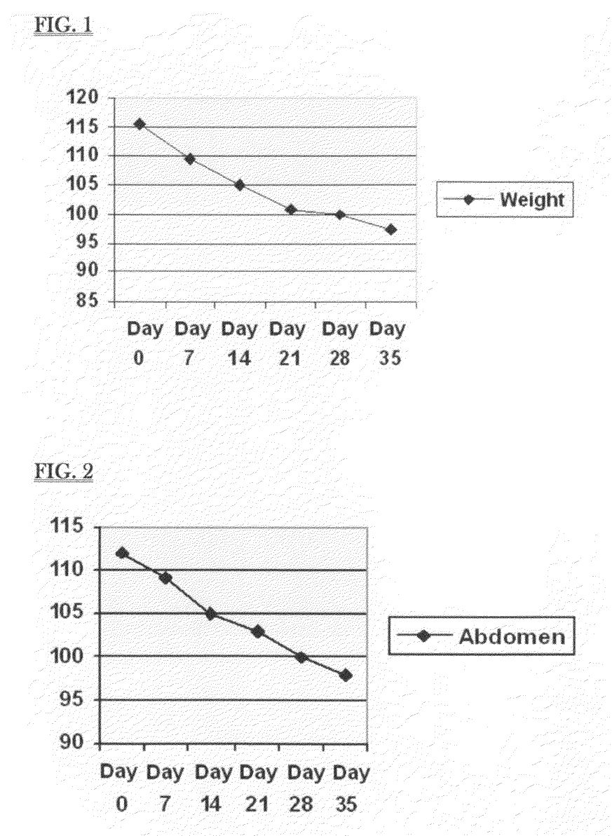

Evolution of the different parameters:
1. Body weight in Kg. (FIG. 1)
2. Abdominal circumference in cm. (FIG. 2)
3. Glycemia in mg/dl (FIG. 3)

Second patient:

Patient AF: Male aged 59 years
Weight: 114.800 kg Height: 1.86 cm
Abdominal obesity (waist circumference): 132 cm
Triglycerides: 313 mg/dl
HDLC: 32 mg/dl
Blood pressure: 160-100 mmHg (administered with antihypertensive medication)
Fast glycemia: 255 mg/dl (administered with oral antidiabetic medication)

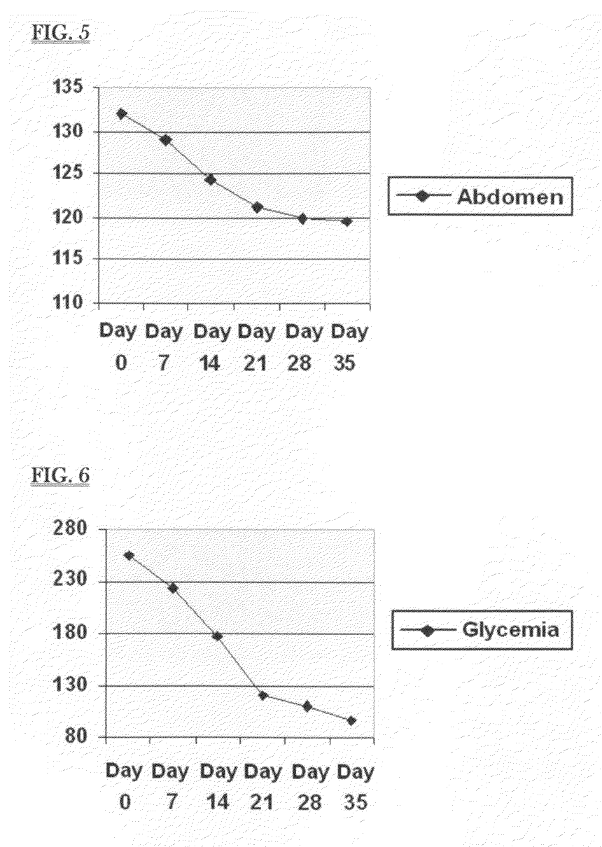

Evolution of the different parameters:
1. Body weight in Kg (FIG. 4)
2. Abdominal circumference in cm (FIG. 5)
3. Glycemia in mg/dl (FIG. 6)

Third patient:

Patient LVM: Female aged 60 years
Weight: 93 Kg
Height: 1.61 cm
Abdominal obesity (waist circumference): 101 cm
Triglycerides: 290 mg/dl
HDLC: 43 mg/dl
Blood pressure: 170-100 mmHg (administered with antihypertensive medication)

Fast glycemia: 194 mg/dl (administered with oral antidiabetic medication)

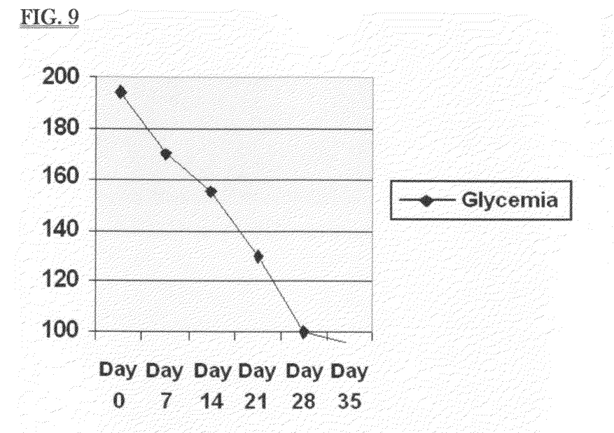

Evolution of the different parameters:
1. Abdominal circumference in cm (FIG. 7)
2. Body weight in Kg. (FIG. 8)
3. Glycemia in mg/dl (FIG. 9)

The advantages of this invention are plain from the description above as well as the images included, showing clear functional advantages of the product, characterizing the invention and representing a beneficial technological improvement that warrants the inclusion of the invention in the law with the pertinent legal protection as per the appended claims.

The nature and scope of this invention as well as its practical use having been described and determined above, it is hereby stated that these are the claims:

1. THE USE OF HUMAN CHORIONIC GONADOTROPIN ORAL OR INJECTABLE FOR THE TREATMENT OF METABOLIC SYNDROME characterized by administration of Human Chorionic Gonadotropin (hCG) in patients with Metabolic Syndrome, in a range of 300-600 International Units daily by oral route, or 100-300 International Units daily by injection during the treatment period.

2. THE USE OF HUMAN CHORIONIC GONADOTROPIN ORAL OR INJECTABLE FOR THE TREATMENT OF METABOLIC SYNDROME of claim 1, characterized by administration of Human Chorionic Gonadotropin (hCG) for the treatment of high blood pressure.

3. THE USE OF HUMAN CHORIONIC GONADOTROPIN ORAL OR INJECTABLE FOR THE TREATMENT OF METABOLIC SYNDROME of claim 1, characterized by administration of Human Chorionic Gonadotropin (hCG) for the improvement of clinical and laboratory findings of diabetes type 2 or of reactive hyperglycemias.

4. THE USE OF HUMAN CHORIONIC GONADOTROPIN ORAL OR INJECTABLE FOR THE TREATMENT OF METABOLIC SYNDROME of claim 1, characterized by administration of Human Chorionic Gonadotropin (hCG) as a hypolipemic agent and hypotriglyceride activity inducer.

5. THE USE OF HUMAN CHORIONIC GONADOTROPIN ORAL OR INJECTABLE FOR THE TREATMENT OF METABOLIC SYNDROME of claim 1, characterized by administration of Human Chorionic Gonadotropin (hCG) as a hypocholesterolemic agent.

6. THE USE OF HUMAN CHORIONIC GONADOTROPIN ORAL OR INJECTABLE FOR THE TREATMENT OF METABOLIC SYNDROME of claim 1, characterized by administration of Human Chorionic Gonadotropin (hCG) as a coadyuvant therapeutic agent for the treatment of gout clinical symptoms.

7. THE USE OF HUMAN CHORIONIC GONADOTROPIN ORAL OR INJECTABLE FOR THE TREATMENT OF METABOLIC SYNDROME of claim 1, characterized by administration of Human Chorionic Gonadotropin (hCG) as a therapeutic agent for the treatment of overweight.

8. THE USE OF HUMAN CHORIONIC GONADOTROPIN ORAL OR INJECTABLE FOR THE TREATMENT OF METABOLIC SYNDROME of claim 1, characterized by administration of Human Chorionic Gonadotropin (hCG) as a therapeutic agent for patients' general condition and for the achievement of a feeling of well being during the treatment period.
